(19) United States
(12) Patent Application Publication
(10) Pub. No.: US 2007/0155973 A1
(43) Pub. Date: Jul. 5, 2007

(54) Novel intermediate compound for the preparation of prostaglandin F analogue

(75) Inventors: Chi-Hsiang Yao, Taoyuan County (TW); Chieh-Li Tung, Taoyuan County (TW)

Correspondence Address: BACON & THOMAS, PLLC, 625 SLATERS LANE, FOURTH FLOOR, ALEXANDRIA, VA, 22314, US

(73) Assignee: Everlight USA, Inc., Pineville, NC (US)

(21) Appl. No.: 11/452,331

(22) Filed: Jun. 14, 2006

(30) Foreign Application Priority Data
Dec. 30, 2005 (CN) 200510003395.1

(51) Int. Cl. C07F 7/02; C07C 67/03

(52) U.S. Cl. 554/77; 554/108

(57) ABSTRACT

A method for the preparation of a prostaglandin F analogue presented by the following formula (I):

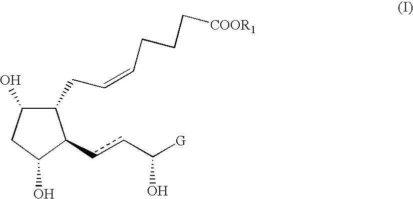

is disclosed, wherein
R₁, G₁, and [symbol] are as defined in the specification. A novel intermediate compound for the preparation of a prostaglandin F analogue is also disclosed.

### BACKGROUND OF THE INVENTION

1. Field of the Invention

The present invention relates to a preparation method for a prostaglandin F analogue. The present invention also relates to a novel intermediate compound for the preparation of prostaglandin F analogue.

2. Description of Related Art

Clinically, prostaglandin F analogues are used for treating glaucoma or high intraocular pressure of other causes. Glaucoma is a disease that the intraocular pressure is discontinuously or continuously elevating, and the chronic high intraocular pressure can damage the tissues in the eyeball as well as patients' vision. If it is not treated in time, the optic nerve may be damaged, followed by the failure of eyesight and deficits of visual field, and the worst cases may lead to blindness. Currently glaucoma is one of the three leading causes of blindness in the industrialized countries. Prostaglandin analogues have excellent curative effect on glaucoma or other ocular hypertension, and therefore the use and the preparation method of prostaglandin analogues have aroused the attention of many chemists and physicians, as documented in U.S. Pat. No.4,599,353, European Patent No. 364417, 495069, 544899, and PCT Patent Publication No. WO95/11003, WO01/055101, WO01/087816, WO02/096868, WO02/096898, WO03/008368.

### SUMMARY OF THE INVENTION

The present invention provides a novel method for the preparation of prostaglandin F analogues.

The present invention also provides a novel intermediate compound for the preparation of prostaglandin F analogues.

The present invention relates to a method for the preparation of the prostaglandin F analogues represented by the following formula (I):

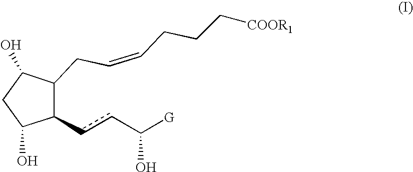

wherein,
R₁ is hydrogen or C₁-C₅ alkyl group;
   G is selected from the group consisting of (i) C₁-C₅ linear alkyl group, (ii) —(CH)₂Ph, and (iii) —CH₂OR^{b}, wherein R^{b} is Cl-substituted benzyl group or CF₃-substituted benzyl group;
[symbol] represents single-bond or double-bond structure, and when being double-bond, it includes both cis- and trans- structure.

The preparation method of the present invention utilizes the novel intermediate of the following formula (7), formula (8), or the mixture of both:

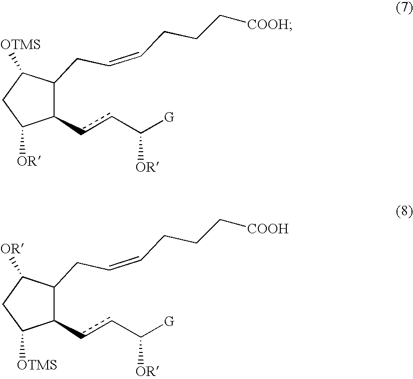

wherein,
G and [symbol] are defined as above;
R′ in both formulas is identical and represents the following substitution group:

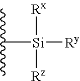

wherein R^{x}, R^{y}, and R^{z} being identical to each other or not, each independently represents C₁-C₆ alkyl group, C₆-C₁₀ aryl group, or C₇-C₁₆ arylalkyl group, while at least one of R^{x}, R^{y}, or R^{z} is not methyl group; to perform the following synthetic reaction (A) or (B), so as to obtain the compound of formula (I) of the present invention.

The synthetic reaction (A) comprises:

(e) reacting the compound of the abovementioned formula (7), formula (8), or the mixture of both with a compound represented by the following formula:

R₁-Z

wherein R₁ is hydrogen or C₁-C₅ alkyl group; Z is halogen, sulphate, mesyl, tosyl, or hydroxyl group; to perform esterification reaction, obtaining a compound represented by the following formula (9), a compound represented by the following formula (10), or a mixture of both,

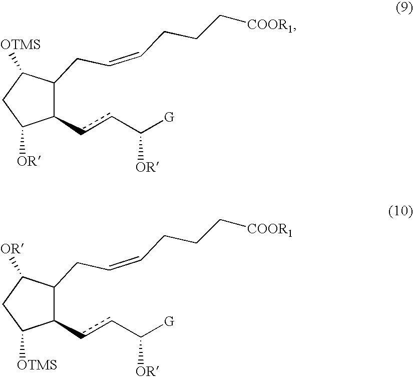

wherein, G, R′, R₁ and [symbol] are defined as above; and

(f) deprotecting the compound of formula (9), (10), or the mixture of both, to obtain the compound represented by formula (I);

the synthetic reaction (B) comprises:

(g) deprotecting the compound represented by formula (7), (8), or the mixture of both, to obtain a compound represented by the following formula (11):

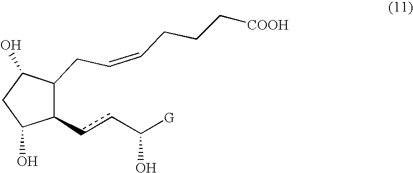

wherein, G and [symbol] are defined as above; and

(h) reacting the compound represented by formula (11) with a compound represented by the following formula:

R₁-Z

wherein R₁ and Z are defined as above; to perform esterification reaction, obtaining the compound of formula (I).

The novel intermediate compounds of formula (7), (8), or the mixture of both of the present invention are prepared by the following steps:

(a) reacting a compound represented by the following formula (2):

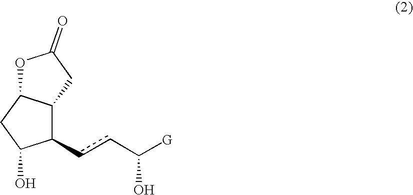

wherein G and [symbol] are as defined in compound (I); with a silylation agent of the following formula:

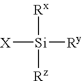

wherein R^{x}, R^{y}, and R^{z} being identical to each other or not, each independently represents C₁-C₆ alkyl group, C₆-C₁₀ aryl group, or C₇-C₁₆ arylalkyl group, while at least one of R^{x}, R^{y} and R^{z} is not methyl group; X is fluorine, chlorine, bromine or iodine; to perform protection reaction, obtaining a compound of the following formula (3):

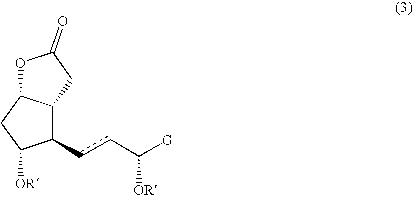

wherein G, R′ and [symbol] are as defined in compound (7), compound (8) or the mixture of both;

(b) reducing the compound of formula (3) to obtain a compound represented by the following formula (4):

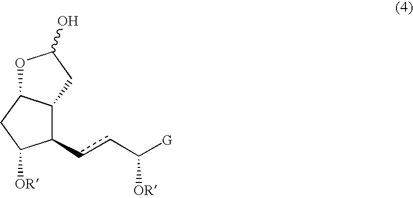

wherein G, R′ and [symbol] are defined as above;

(c) reacting the compound of formula (4) with the following compound:

HOOC(CH₂)₄P⁺(R^{a})₃Y⁻

wherein R^{a} is C₁-C₆ alkyl group or C₆-C₁₀ aryl group; Y is fluorine, chlorine, bromine or iodine; to perform Wittig Reaction, obtaining a compound represented by the following formula (5), a compound represented by the following formula (6), or a mixture of both:

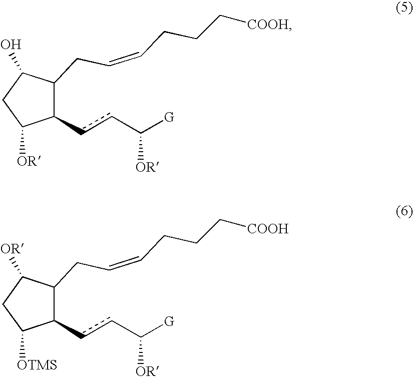

wherein G, R′ and [symbol] are defined as above; and

(d) reacting the compound of formula (5), (6) or the mixture of both with the following silylation agent:

Me₃Si—X

wherein X is fluorine, chlorine, bromine or iodine; to perform protection reaction, obtaining the compound represented by formula (7), (8) or the mixture of both.

In the method for the preparation of the compound of formula (I) according to the present invention, the R₁ substitution group in formula (I) is preferably hydrogen or isopropyl group. The G substitution group is preferably selected from the group consisting of

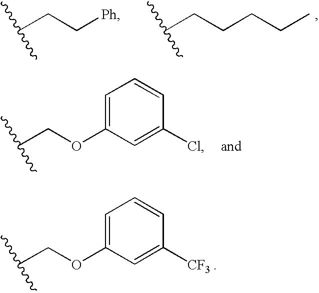

According to the present invention, examples of the compound of formula (I) obtained from the preparation method of compound of formula (I) include:

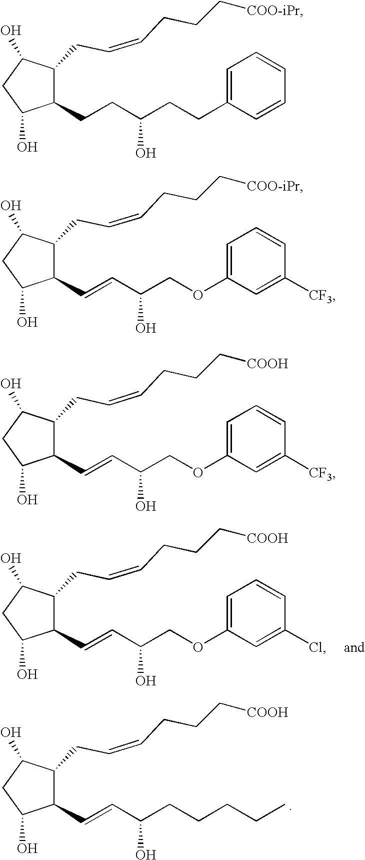

The present invention also relates to the abovementioned novel intermediate as compound of formula (7) or (8).

Examples of compound (7) include the compound represented by the following formula (7a):

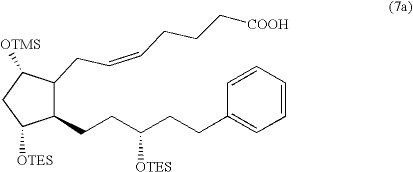

wherein TMS is trimethyl silyl; TES is triethyl silyl.

Examples of compound (8) include the compound represented by the following formula (8a):

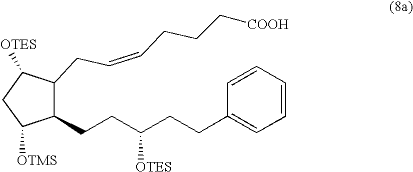

wherein TMS is trimethyl silyl; TES is triethyl silyl.

The present invention also relates to the abovementioned novel intermediate as compound of formula (9) or (10).

Examples of compound (9) include the compound represented by the following formula (9a):

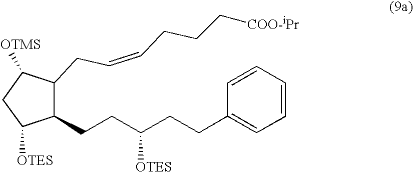

wherein TMS is trimethyl silyl; TES is triethyl silyl.

Examples of compound (10) include the compound represented by the following formula (10a):

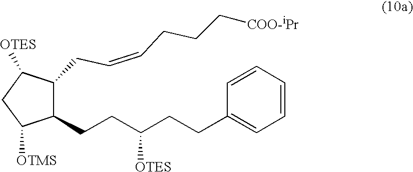

wherein TMS is trimethyl silyl; TES is triethyl silyl.

### DETAILED DESCRIPTION OF THE PREFERRED EMBODIMENT

According to the present invention, the method for the preparation of the prostaglandin F analogues represented by the following formula (I):

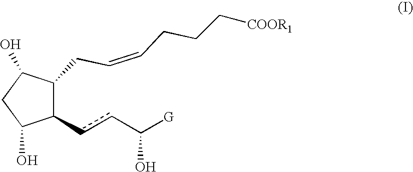

wherein,
R₁ is hydrogen or C1-C5 alkyl group;
G is selected from (i) C1-C5 linear alkyl group, (ii) —(CH)₂Ph, and (iii) —CH₂OR^{b}, wherein R^{b} is Cl- or CF₃-substituted benzyl group;
[symbol] represents single-bond or double-bond structure, and when being double-bond, it includes both cis- and trans-structure;

utilizes the novel intermediate of the following formula (7), formula (8), or the mixture of both:

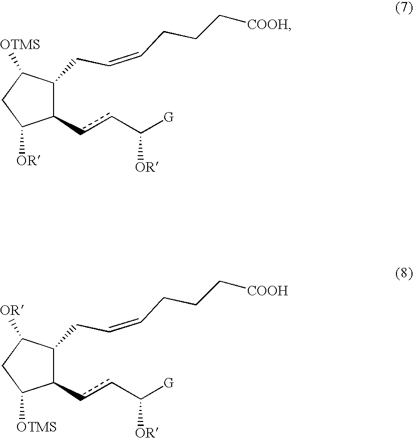

wherein,
G, R′ and [symbol] are defined as above; to perform synthetic reactions.

The synthesis process of compound (I) is shown in the following Scheme 1.

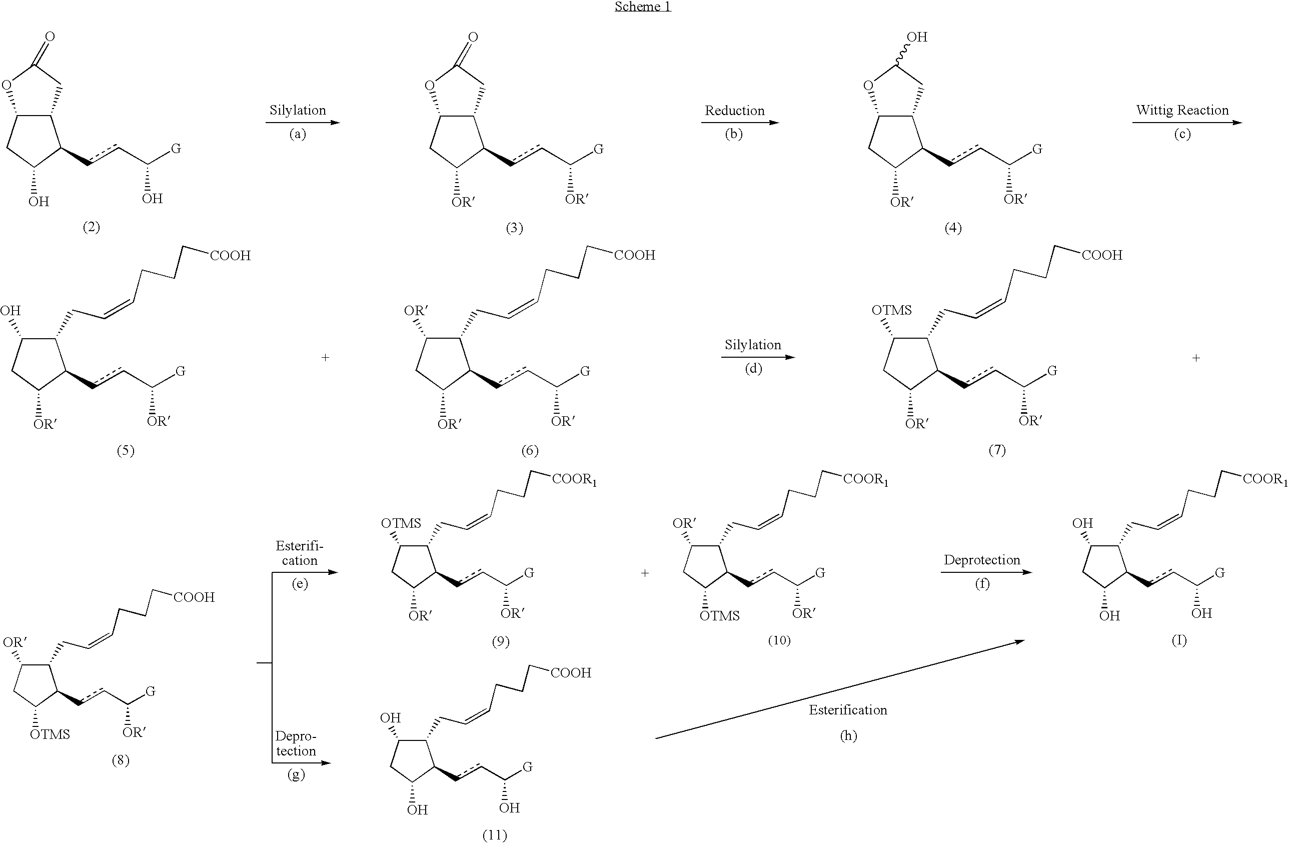

wherein, G, R′, R₁, and [symbol] are defined as above; TMS is trimethyl silyl.

The synthesis process of the prostaglandin F analogue as formula (I) can be illustrated as follows:

(a) protecting the compound of formula (2) to obtain the compound of formula (3):

in the preparation process, the compound of formula (2)

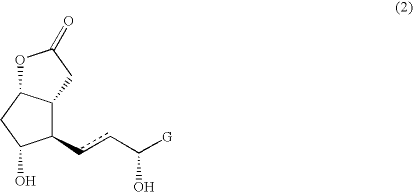

wherein G and [symbol] are defined as above; is protected under basic condition in organic solvents. This reaction starts with adding alkaline reagents, followed by the addition of a silylation agent of the following formula:

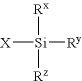

wherein, R^{x}, R^{y}, and R^{z} being identical to each other or not, each independently represents C₁-C₆ alkyl group, C₆-C₁₀ aryl group, or C₇-C₁₆ arylalkyl group, while at least one of R^{x}, R^{y} and R^{z} is not methyl group; X is fluorine, chlorine, bromine or iodine; to obtain the compound of the following formula (3):

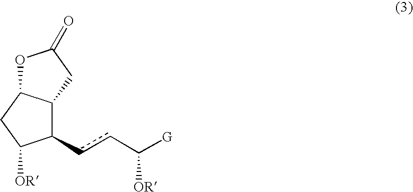

wherein G, R′ and [symbol] are defined as above.

In this reaction, the organic solvents are the polar solvents known to the art, for example, tetrahydrofuran (THF), dimethyl formamide (DMF), dimethyl sulfoxide (DMSO), toluene, diethyl ether, dichloromethane, or dichloroethane, and among these, the solvents with medium to high polarity such as THF, DMF, toluene or diethyl ether, are preferably used.

Examples of the alkaline reagents include triethylamine, diisopropylethylamine, or 1,8-diazabicyclo[5,4,0]undec-7-ene (DBU), and triethylamine is preferably used.

Examples of the silylation agents include triethylsilyl chloride, tert-butyldimethylsilyl chloride, tert-butyldiphenylsilyl chloride, or phenyldimethylsilyl chloride, and triethylsilyl chloride is preferably used.

The temperature of the reaction ranges from 30˜−10° C., and preferably ranges from 0˜5° C.

(b) reducing the compound of formula (3) to obtain the compound of formula (4):

in the preparation process, reducing agent is added to the compound of formula (3) in organic solvents under low temperature, so as to reduce the lactone group to lactol group, and thus the compound of the following formula (4):

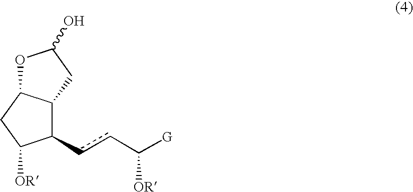

wherein, G, R′ and [symbol] are defined as above; can be obtained.

In the reaction, the organic solvents are those polar solvents known to the art, for example, tetrahydrofuran (THF), toluene, diethyl ether, dichloromethane, or dichloroethane, and among these, THF, toluene or diethyl ether is preferably used; THF or toluene is most preferably used.

Examples of reducing agent include diisobutylaluminum hydride (DIBAL-H). The temperature of the reaction ranges between −60˜80° C., and preferably between −60˜70° C.

(c) performing Wittig Reaction with the compound of formula (4) to obtain compound of formula (5) or (6) or the mixture of both:

In the preparation process, alkaline reagents are added to compound (4) along with the compound of the following formula:

HOOC(CH₂)₄P⁺(R^{a})₃Y⁻

wherein R^{a} is C₁-C₆ alkyl group or C₆-C₁₀ aryl group; Y is fluorine, chlorine, bromine or iodine; in organic solvents, and the Wittig Reaction is performed to obtain the compound of the following formula (5) or (6) or the mixture of both,

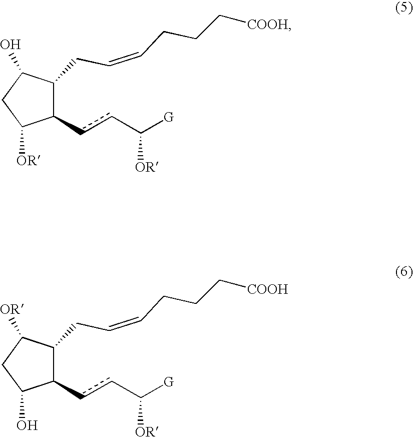

wherein, G, R′ and [symbol] are defined as above.

In this reaction, the organic solvents are high-polar solvent, medium-polar solvent, or chlorinated solvent known to the art, for example, tetrahydrofuran (THF), toluene, dichloromethane, dichloroethane, or ester type solvents, and among these, THF or toluene is preferably used; THF is most preferably used.

The alkaline reagents used in the reaction can be organic or inorganic alkali. Examples of the alkaline reagents include triethylamine, diisopropylethylamine, 1,8-diazabicyclo[5.4.0]undec-7-ene (DBU), sodium hydride(NaH), potassium carbonate (K₂CO₃), or potassium tert-butoxide, and among these, potassium tert-butoxide, triethylamine, potassium carbonate or sodium hydride is preferably used; potassium tert-butoxide is most preferably used.

The temperature of the reaction is generally between −20˜40° C., and preferably between 0˜5° C.

(d) protecting compound (5) or (6) or the mixture of both to obtain compound (7) or (8) or the mixture of both:

In the process, compound (5) or (6) or the mixture of both directly undergoes protection reaction under basic condition in the organic solvents. The alkaline reagents is added first, followed by a silylation agent of the following formula,

Me₃Si—X

wherein, X is fluorine, chlorine, bromine or iodine; to perform protection reaction, obtaining the compound of the following formula (7) or (8) or the mixture of both:

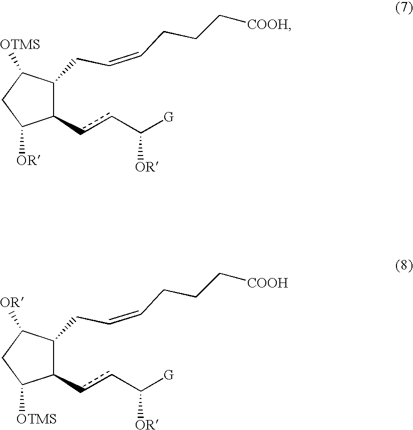

wherein, G, R′ and [symbol] are defined as above.

In the reaction, the organic solvents are the polar solvents known to the art, for example, tetrahydrofuran (THF), dimethyl formamide (DMF), dimethyl sulfoxide (DMSO), toluene, diethyl ether, dichloromethane, or dichloroethane, and among these, the solvent with medium to high polarity such as THF, DMF, toluene or diethyl ether, is preferably used.

Examples of the alkaline reagents include triethylamine, diisopropylethylamine, or 1,8-diazabicyclo[5,4,0]undec-7-ene (DBU), and triethylamine is preferably used.

Examples of the silylation agent include trimethylsilyl chloride.

The temperature of the reaction is generally between 30˜−10° C., and preferably between 0˜−5° C.

(e) performing esterification with compound (7) or (8) or the mixture of both to obtain the compound of the following formula (9) or (10) or the mixture of both:

in the preparation process, after adding acidic or basic catalysts to compound (7) or (8) or the mixture of both in organic solvents, the compound of the following formula,

R₁-Z

wherein R₁ is defined as above; Z is halogen, sulphate, mesyl, tosyl or hydroxyl group; is then added to perform esterification, obtaining the compound of the following formula (9) or (10) or the mixture of both:

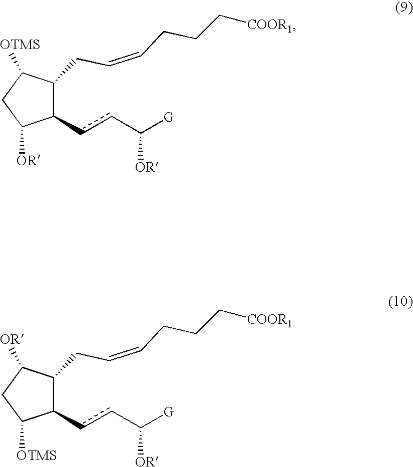

wherein, G, R′, R₁ and [symbol] are defined as above.

In the reaction, the organic solvents are the polar solvents known to the art, for example, tetrahydrofuran (THF), dimethyl formamide (DMF), dimethyl sulfoxide (DMSO), toluene, diethyl ether, dichloromethane, dichloroethane, methanol, ethanol, isopropanol, or acetone, and among these, THF, DMF, alcohols or acetone is preferably used. The catalysts can be organic acids or bases, for example, triethylamine, diisopropylethylamine, pyridinium p-toluenesulfonate (PPTS), p-Toluene sulfonic acid (PTSA) or 1,8-diazabicyclo[5,4,0]undec-7-ene (DBU), and triethylamine, diisopropylethylamine, or DBU is preferably used.

The temperature of the reaction is generally between 40˜−10° C., and preferably between 20˜25° C.

(f) deprotecting compound (9) or (10) or the mixture of both to obtain compound (I):

in the preparation process, acidic catalysts are added to the organic solvents, aqueous solution, or mixture of organic solvents and aqueous solution in different proportions that contains compound (9) or (10) or the mixture of both so as to carry out the deprotection reaction, and thus compound (I) is obtained.

In the reaction, the catalysts can be inorganic and organic acid, for example, pyridinium p-toluenesulfonate (PPTS), p-Toluene sulfonic acid (PTSA), hydrochloric acid, or acetic acid, and among these, pyridinium p-toluenesulfonate (PPTS), p-Toluene sulfonic acid (PTSA) or hydrochloric acid is preferably used.

The temperature of the reaction is generally between 40˜−10° C., and preferably between 0˜5° C.

On the other hand, other than using compound (7) or (8) or the mixture of both to carry out the reaction, compound (I) can be obtained through other synthetic route, for example:

(g) deprotecting compound (7) or (8) to obtain compound of the following formula (11):

In the preparation process, acidic catalysts are added to the organic solvents, aqueous solution, or mixture of organic solvents and aqueous solution in different proportions that contains compound (7) or (8) or the mixture of both so as to carry out the deprotection reaction, and thus the compound of the following formula (11):

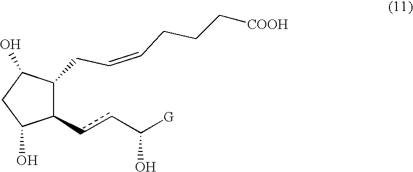

wherein, G and [symbol] are defined as above; can be obtained.

In the reaction, the organic solvents are the polar solvents known to the art, for example, tetrahydrofuran (THF), methanol, ethanol, isopropanol, or acetone, and among these, THF, alcohols or acetone is preferably used.

The catalysts can be inorganic and organic acid, for example, pyridinium p-toluenesulfonate (PPTS), p-Toluene sulfonic acid (PTSA), hydrochloric acid, or acetic acid, and among these, pyridinium p-toluenesulfonate (PPTS), p-Toluene sulfonic acid (PTSA) or hydrochloric acid is preferably used.

The temperature of the reaction is generally between 40˜−10° C., and preferably between 0˜5° C.

(h) performing further esterification with compound (11) to obtain compound (I):

In the preparation process, acidic or basic catalysts are added to compound (11) along with the following compound:

R₁-Z

wherein, R₁ and Z are defined as above; in organic solvents, and then esterification is carried out to obtain the prostaglandin F analogue of formula (I).

In the reaction, the organic solvents are the polar solvents known to the art, for example, tetrahydrofuran (THF), dimethyl formamide (DMF), dimethyl sulfoxide (DMSO), toluene, diethyl ether, dichloromethane, dichloroethane, methanol, ethanol, isopropanol, or acetone, and among these, THF, DMF, alcohols or acetone is preferably used. The catalysts can be organic acids or bases, for example, triethylamine, diisopropylethylamine, pyridinium p-toluenesulfonate (PPTS), p-Toluene sulfonic acid (PTSA) or 1,8-diazabicyclo[5,4,0]undec-7-ene (DBU), and triethylamine, diisopropylethylamine, or DBU is preferably used.

The temperature of the reaction is generally between 40˜−10° C., and preferably between 20˜25° C.

In the synthetic process of the present invention, two different silylation agents are used to carry out the reaction. In the first silylation reaction such as step (a), compound (2) is reacted with silylation agent to perform protection reaction, so as to prevent the unprotected hydroxyl group on the carbon chain from reacting with the reducing agent in the following reduction reaction that may decrease the yield. Therefore, those with larger and harder-to-hydrolyze silyl group, for example, triethylsilyl chloride, tert-butyldimethylsilyl chloride, tert-butyldiphenylsilyl chloride, or phenyldimethylsilyl chloride, were chosen for the protection reaction. In the second silylation reaction such as step (d), compound (5) or (6) or the mixture of both is reacted with the smaller and easier-to-hydrolyze trimethylsilyl chloride, so as to increase the lipophilicity of the compound (7) or (8) or the mixture of both obtained from the second silylation reaction. Therefore, after removing the impurity with high polarity in aqueous layer, such as phosphonium oxide acid derivatives, by extraction, followed by esterification and deprotection reaction, the prostaglandin F analogue of formula (I) can be obtained.

The preparation of compound (2) in Scheme 1 can be accomplished through the synthesis methods mentioned in several documents, for example, European Patent No. 364417, Zhongguo Yaowu Huaxue Zazhi (1998), 8(3), 213-217, or Taiwan Patent Application No. 93131143, so that compound (2) can be synthesized. The synthetic routes of prostaglandin analogues stated in the abovementioned patent documents can be illustrated in the following Scheme 2:

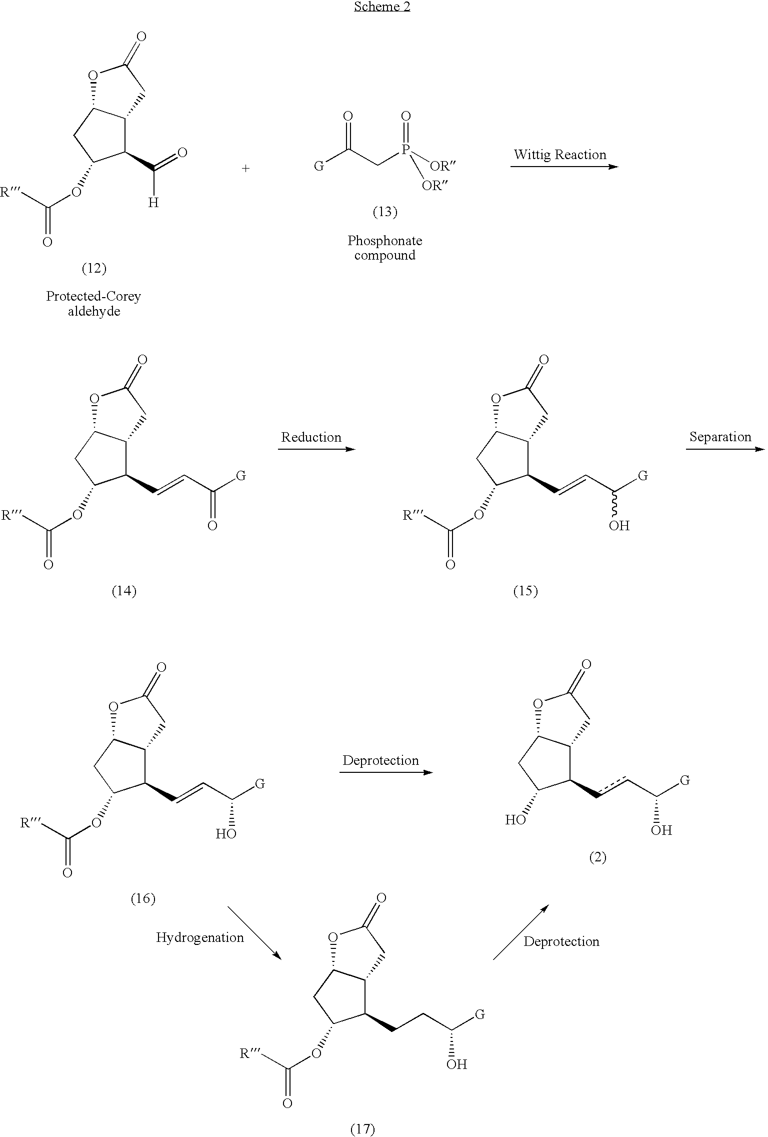

wherein, G is selected from (i) C₁-C₅ linear alkyl group, (ii) —(CH)₂Ph, and (iii) —CH₂OR^{b}, wherein R^{b} is Cl- or CF₃-substituted benzyl group;
R″ is C₁-C₆ alkyl group;
R′″ is C₆-C₁₀ aryl group that contains 0˜3 substitution groups, wherein those substitution groups are selected from halogen, C₁-C₆ alkyl group, or C₆-C₁₀ aryl group;
[symbol] represents single-bond or double-bond structure, and when being double-bond, it includes both cis- and trans-structure.

The present invention will be further illustrated in the following examples. Unless otherwise stated, the “%” used in the examples refers to weight percentage, and the temperature is in ° C.

### EXAMPLE 1

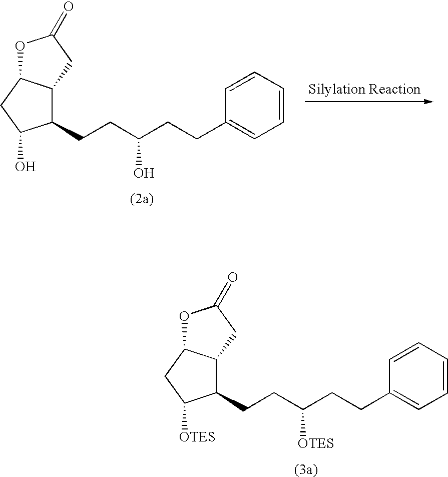

3.9 g of imidazole, 17.6 g of compound (2a), and 200 ml of DMF were added to a 1000 ml three-necked flask. Under nitrogen atmosphere and temperature between 0-5° C., 17.5 g of triethylamine was added dropwise, followed by stirring for 0.5 hr. Then, under nitrogen atmosphere, 24.3g of triethylsilyl chloride was added dropwise, followed by stirring for another 0.5 hr, and the completion of reaction was checked by TLC. After the completion of reaction, 250 g of n-hexane was added for extraction. The top layer was extracted and then dehydrated with sodium sulfate. After filtering out the sodium sulfate, the filtrate was vacuum condensed to give 36.8 g of yellow oil. (compound (3a))

¹H NMR (CDCl₃) : δ: 7.35-7.12 (m,5H), 4.94 (dt,1H), 3.92 (q,1H), 3.69 (m,1H), 2.59-2.44 (m,3H), 2.18-2.07 (m,2H), 1.99 (d,1H), 1.85-1.68 (m,5H), 1.58-1.43 ( m,2H ), 1.43-1.32 (m,1H), 1.01-0.86 (m,18H), 0.66-0.45(m,12H).

¹³C NMR (CDCl₃): δ: 177.42, 142.26, 128.31, 128.19, 125.71, 83.91, 77.49, 71.67, 55.23, 42.63, 40.62, 38.87, 36.16, 35.16, 31.71, 28.66, 6.34, 2.73

### EXAMPLE 2

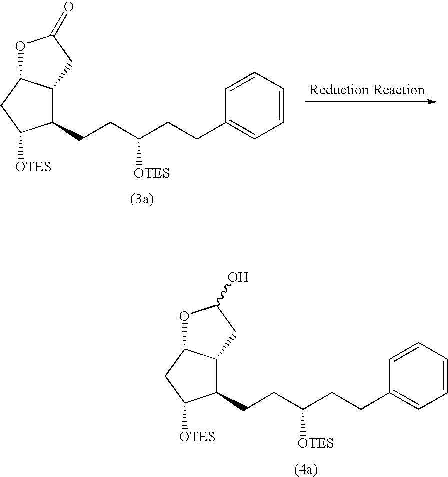

36.8 g of compound (3a), and 300 ml of toluene were added to a 1000 ml three-necked flask. Under nitrogen atmosphere, the temperature was reduced to —60˜70° C., and then 67.0 g of diisobutylaluminum hydride (DIBAL-H, 1M, D=0.7) was added dropwise, followed by stirring for 0.5 hr. The completion of reaction was checked by TLC. After the completion of reaction, the dry-ice bath was removed and then 270 ml of saturated sodium sulfate solution, followed by stirring for 30 min. After filtering with celite, the filtrate was extracted with 200 ml of water. The top layer was extracted and then dehydrated with sodium sulfate. After filtering out the sodium sulfate, the filtrate was vacuum condensed to yield 36.0 g of yellow oil. (compound (4a))

¹H NMR (CDCl₃): δ: 7.33-7.11 (m,5H), 5.64-5.60 (d,1H), 4.70-4.54 (m,1H), 3.80-3.64 (m,2H), 2.78-2.50 (m,2H), 2.46-2.20 (m,3H), 2.20-1.86 (m,4H) 1.80-1.30 (m,6H), 1.02-0.82 (m,18H), 0.68-0.45(m,12H)

¹³C NMR (CDCl₃): δ: 142.54, 128.35, 128.26, 125.64, 100.50, 80.66, 78.60, 72.12, 54.06, 44.75, 41.34, 40.89, 38.88, 35.26, 31.79, 28.75, 6.57, 5.15

### EXAMPLE 3

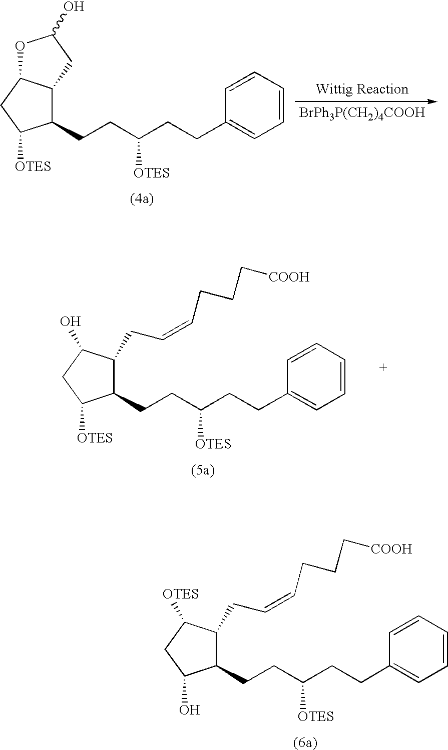

3.80 g of 4-carboxybutyltriphenylphosphonium bromide and 15 ml of THF were added to a 100 ml three-necked flask. After reducing the temperature to 0˜5° C., 2.89 g of potassium tert-butoxide was added, and ylide of orange color was obtained. After stirring for 1 hour, compound (4a) in THF solution (2.0 g of compound (4a) dissolved in 150 ml of THF) was added and kept stirring for another 1 hour before checking the completion of reaction by TLC. After the reaction was completed, went straight to the next step.

¹H NMR (CDCl₃): δ: 7.30-7.11 (m,4H), 6.98 (s,1H), 5.45-5.23 (m,2H), 4.12-4.04 (q,1H), 3.78-3.64 (m,2H), 2.76-2.48 (m,2H), 2.36-1.98 (m,6H), 1.80-1.30 (m,12H), 1.02-0.84 (m,18H), 0.67-0.44(m,12H).

¹³C NMR (CDCl₃): δ: 179.16, 142.64, 129.60, 128.62, 128.46, 128.27, 125.59, 76.27, 72.49, 71.80, 50.14, 48.19, 44.27, 39.12, 35.17, 34.26, 31.74, 27.87, 26.98, 25.76, 25.47, 6.65, 4.94

### EXAMPLE 4

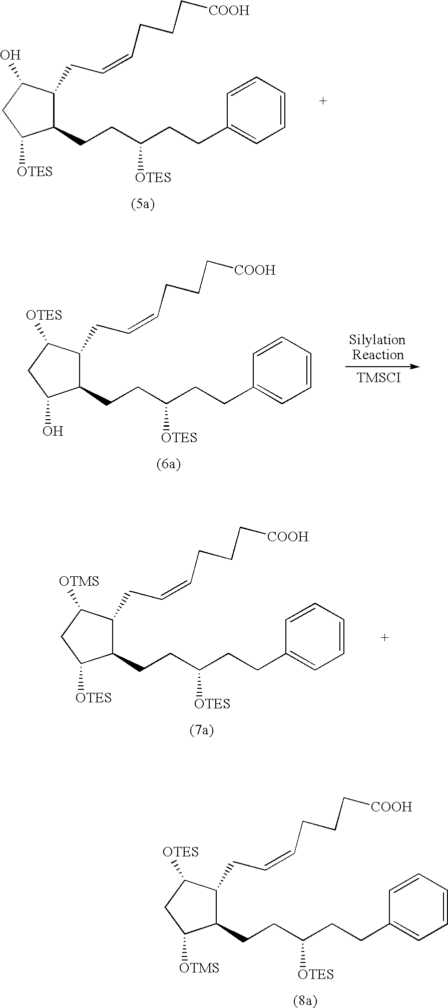

0.25 g of imidazole was added to the reaction flask, and under nitrogen atmosphere and temperature between 0˜5° C., 1.33 g of triethylamine was added dropwise, followed by stirring for 0.5 hr. Under nitrogen atmosphere, 1.36 g of trimethylsilyl chloride was then added and stirred for another 0.5 hr. The completion of reaction was checked by TLC. After the completion of reaction, 10 ml of NaHCO₃ and 20 g of n-hexane were added for extraction. The top layer was extracted and dehydrated with sodium sulfate. After filtering out the sodium sulfate, the filtrate was vacuum condensed to remove excess solvent, and 100 g of n-hexane was then added while the temperature reduced to between 0˜5° C. Stirring was maintained under the same temperature for 8 hours, and the resulting precipitate was filtered out, and 1.91 g of the mixture of compound (7a) and (8a), a yellow oil, was then obtained.

¹H NMR (CDCl₃): δ: 7.24-7.01 (m,5H), 5.40-5.20 (m,2H), 4.04-3.96 (m,1H), 3.72-3.52 (m,2H), 2.63-2.44 (m,2H), 2.24˜1.90 (m,6H), 1.76-1.15(m,12H), 1.00-0.72 (m,18H), 0.61-0.44(m,12H), 0.10˜−0.04(s, 9H).

¹³C NMR (CDCl₃): δ: 178.89, 142.55, 130.22, 128.63, 128.28, 128.25, 125.61, 76.35, 72.65, 71.59, 50.07, 48.01, 44.17, 39.08, 34.04, 33.45, 31.78, 27.49, 26.58, 25.62, 24.69, 6.87, 4.92, 0.12

### EXAMPLE 5

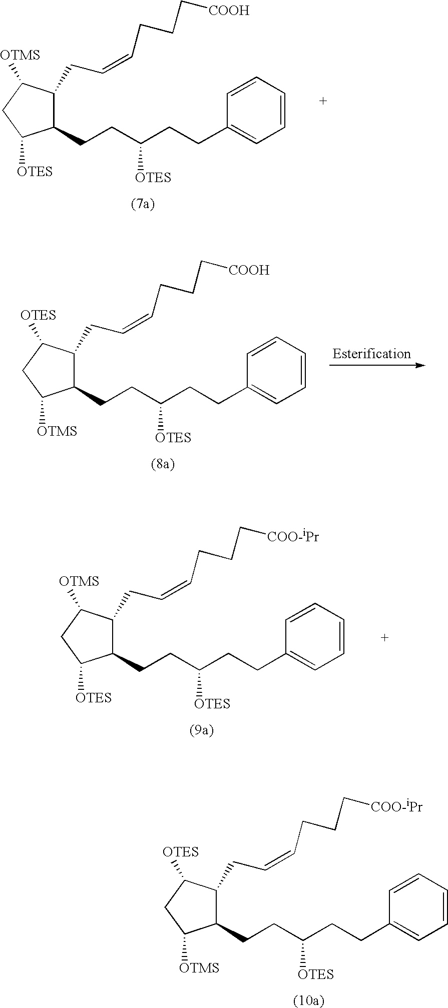

0.41 g of the mixture of compound (7a) and (8a) was dissolved in 20 ml of acetone and under 20˜25° C., 0.59 g of 1,8-diazabicyclo[5.4.0]undec-7-ene was added, followed by 10 min of stirring. 0.43 g of 2-bromopropane was then added and stirring was maintained for 0.5 hr. The completion of reaction was checked by TLC. After the reaction was completed, pH was adjusted between 6.0˜7.0 with 32% hydrochloric acid, followed by the addition of 100 ml of water. Acetone was removed by vacuum suction, and extraction was performed by using ethyl acetate. The top layer was then extracted and dehydrated with sodium sulfate. After filtering out the sodium sulfate, the filtrate was vacuum condensed to obtain 0.69 g of crude product. After purification by column chromatography, 0.36 g the mixture of yellow oil compound (9a) and (10a) were obtained.

¹H NMR (CDCl₃): δ: 7.25-7.04 (m,5H), 5.54-5.22 (m,2H), 4.90 (m,1H), 4.21-3.48 (m,3H) 2.78-2.44 (m,2H), 2.39-1.91 (m,6H), 1.84-1.38 (m,12H), 1.13 (d,6H) 1.00-0.63 (m,27H), 0.60-0.38 (m,12H)

### EXAMPLE 6

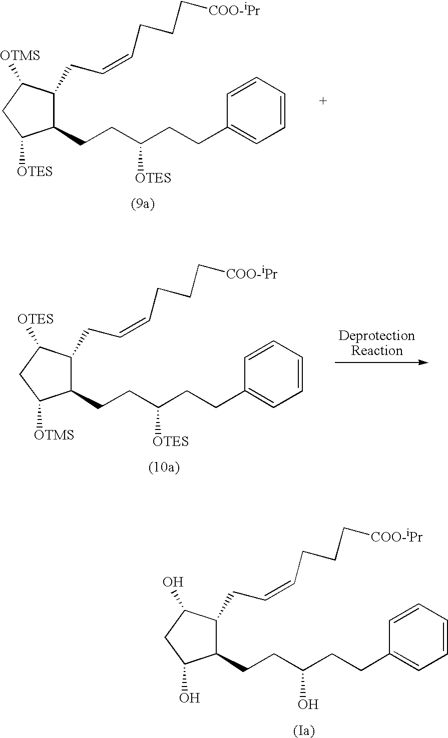

0.36 g of the mixture of compound (9a) and (10a) was dissolved in 15 ml of acetone, to which 15 g of water was then added. pH was adjusted to 1.0˜3.0 with hydrochloric acid under 0˜5° C. The solution was then stirred under 20˜25° C., and the completion of reaction was checked by TLC. After the reaction was completed, acetone was removed by vacuum suction, followed by the addition of 5 ml of water and 20 ml of ethyl acetate for extraction. The top layer was then extracted, and the ethyl acetate was removed by vacuum suction. 50 g of ACN and 50 g of n-hexane were added for extraction, and the bottom ACN layer was extracted and dehydrated with sodium sulfate, which was then filtered out and the filtrate was vacuum condensed to yield 0.19 g of yellow oil. The oil was then purified by column chromatography and 0.17 g of compound (1a), a light yellow oil-like material, was obtained.

Rf=0.35(silica gel, EA/Hx=7/3)

[a]D²⁰=+31.82 (C=0.9, Acetonitrile)

¹H NMR (CDCl₃): δ: 7.25 (m,2H), 7.17 (m,2H), 7.15 (m,1H), 5.44 (m,1H), 5.36 (m,1H), 4.97 (m,1H), 4.10 (m,1H), 3.92 (m,1H), 3.63 (m,1H), 2.78 (m,1H), 2.64 (m,1H), 2.28 (m,2H), 2.24 (t, 2H) 2.08 (m,2H), 1.83 (m,2H), 1.74 (m,2H), 1.67 (m,1H), 1.65 (m,2H), 1.58 (m,2H), 1.51 (m,1H), 1.33 (m,1H), 1.28 (m,1H), 1.21 (d,6H)

¹³C NMR (CDCl₃): δ: 173.51, 142.09, 129.49, 129.34, 128.36, 125.76, 78.67, 74.55, 71.26, 67.64, 52.71, 51.79, 42.46, 38.99, 35.74, 34.03, 32.08, 29.64, 26.82, 26.58, 24.89, 21.79

MS: m/z=455 (M+Na)

### EXAMPLE 7

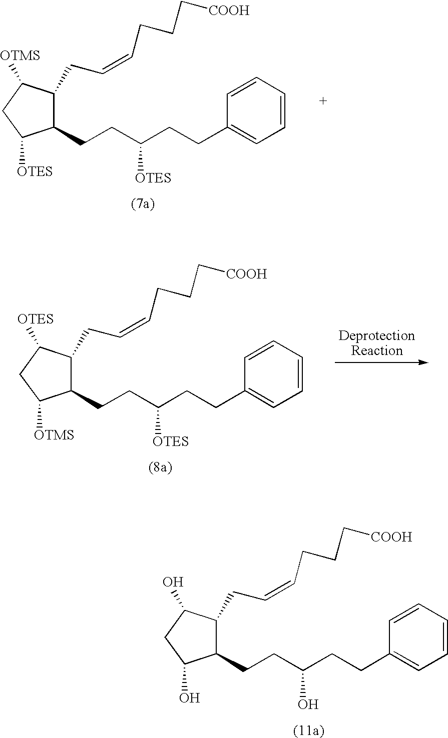

0.39 g of the mixture of compound (7a) and (8a) was dissolved in 15 ml of acetone and 15 g of water was then added. pH was adjusted to between 1.0˜3.0 with 32% hydrochloric acid under 0˜5° C., and stirring was maintained under 20˜25° C. The completion of reaction was checked by TLC. After the reaction was completed, Acetone was removed by vacuum suction, and extraction was performed by the addition of 5 ml of water and 30 ml of ethyl acetate. The top layer was then extracted and dehydrated with sodium sulfate. After filtering out the sodium sulfate, the filtrate was vacuum condensed to give 0.35 g of yellow oil. The yellow oil was then purified by column chromatography, and 0.14 g of compound (11a), a yellow oil, was obtained.

¹H NMR (Methanol-D₄): δ: 7.30-7.08 (m,5H), 5.45 (m.1H), 5.37 (m,1H), 4.15 (b,1H), 3.95 (b,1H), 3.65 (m,1H), 2.77 (m,1H), 2.62 (m,1H), 2.36-1.22 (m,18H)

¹³C NMR (CDCl₃): δ: 177.00, 142.00, 129.30, 129.20, 128.22, 128.19, 125.58, 78.08, 3.93, 71.24, 51.85, 51.22, 42.24, 38.63, 35.07, 33.93, 33.08, 31.91, 8.88, 26.23, 24.52

### EXAMPLE 8

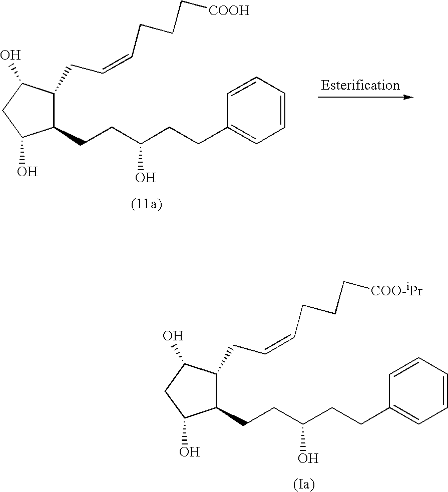

0.14 g of compound (11a) was dissolved in 15 ml of acetone, and under 20˜25° C., 0.35 g of 1,8-diazabicyclo[5.4.0]undec-7-ene was added, followed by 10 min of stirring. 0.43 g of 2-bromopropane was then added and stirring was maintained for 0.5 hr. The completion of reaction was checked by TLC. After the reaction was completed, pH was adjusted to between 6.0˜7.0 using 32% hydrochloric acid, followed by the addition of 5 ml of water. Acetone was removed by vacuum suction, and extraction was performed by using ethyl acetate. The top layer was then extracted and dehydrated with sodium sulfate. After filtering out the sodium sulfate, the filtrate was vacuum condensed to yield 0.20 g of yellow oil. The yellow oil-like material was then purified by column chromatography, and 0.13 g of compound (1a), a light yellow oil, was obtained.

Although the present invention has been explained in relation to its preferred embodiment, it is to be understood that many other possible modifications and variations can be made without departing from the scope of the invention as hereinafter claimed.

What is claimed is:

1. A method for preparing a compound represented by the following formula (I): 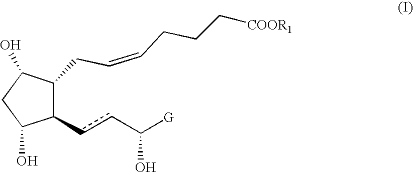
wherein,
R₁ is hydrogen or C₁-C₅ alkyl group;
G is selected from the group consisting of (i) C₁-C₅ linear alkyl group, (ii) —(CH)₂Ph, and (iii) —CH₂OR^{b}, wherein R^{b} is Cl- or CF₃-substituted benzyl group; [symbol]
represents single-bond or double-bond structure, and when being double-bond, it includes both cis- and trans-structure; which comprising the following synthetic reaction (A) or (B):
wherein, the synthetic reaction (A) comprises:
(e) reacting a compound of the following formula (7), a compound of the following formula (8), or a mixture of both 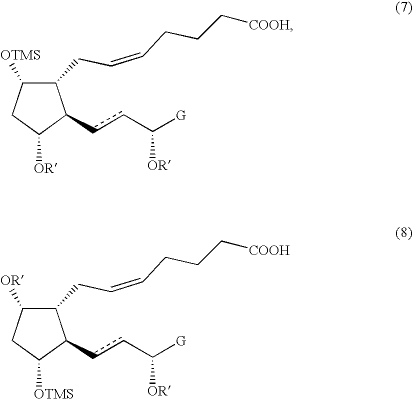
wherein,
G and [symbol] are defined as above;
R′ in both formulas is identical and represents the following substitution group: 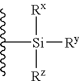
wherein R^{x}, R^{y}, and R^{z} being identical to each other or not, each independently represents C₁-C₆ alkyl group, C₆-C₁₀ aryl group, or C₇-C₁₆ arylalkyl group, while at least one of R^{x}, R^{y}, or R^{z} is not methyl group; with a compound represented by the following formula:
R₁-Z
wherein R₁ is hydrogen or C1-C5 alkyl group; Z is halogen, sulphate, mesyl, tosyl, or hydroxyl group; to perform esterification reaction, obtaining a compound represented by the following formula (9), a compound represented by the following formula (10), or a mixture of both, 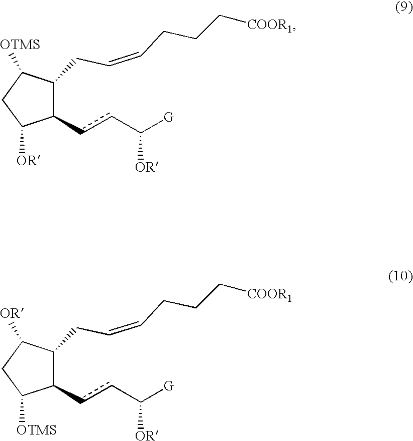
wherein, G, R′, R₁ and [symbol] are defined as above; and
(f) deprotecting the compound represented by formula (9), (10), or the mixture of both, to obtain the compound represented by formula (I);
wherein, the synthetic reaction (B) comprises:
(g) deprotecting the compound represented by formula (7), (8), or the mixture of both, to obtain a compound represented by the following formula (11): 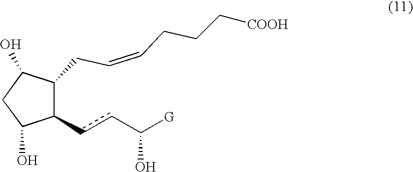
wherein, G and [symbol] are defined as above; and
(h) reacting the compound represented by formula (11) with a compound represented by the following formula:
R₁-Z
wherein and R₁ and Z are defined as above; to perform esterification reaction, obtaining the compound of formula (I).

2. The method of claim 1, wherein the compound represented by formula (7), the compound represented by formula (8), or the mixture of both is prepared by the following steps:
(a) reacting a compound represented by the following formula (2): 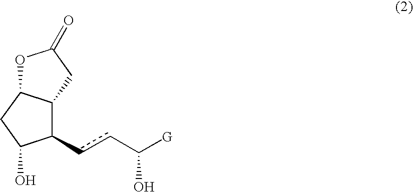
wherein G and [symbol] are as defined in claim 1; with a silylation agent represented by the following formula: 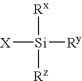
wherein R^{x}, R_{y}, and R^{z} being identical to each other or not, each independently represents C₁-C₆ alkyl group, C₆-C₁₀ aryl group, or C₇-C₁₆ arylalkyl group, while at least one of R^{x}, R^{y} and R^{z} is not methyl group; X is fluorine, chlorine, bromine or iodine; to perform protection reaction, obtaining a compound represented by the following formula (3): 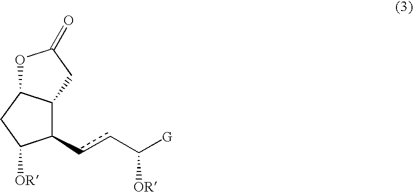
wherein G, R′ and [symbol] are as defined in claim 1;
(b) reducing the compound represented by formula (3) to obtain a compound represented by the following formula (4): 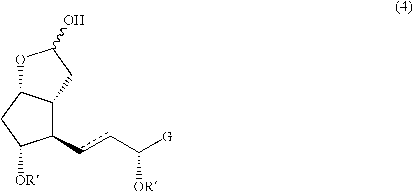
wherein G, R′ and [symbol] are as defined in claim 1;
(c) reacting the compound represented by formula (4) with the following compound:
HOOC(CH₂)₄P⁺(R^{a})₃Y⁻
wherein R^{a} is C₁-C₆ alkyl group or C₆-C₁₀ aryl group; Y is fluorine, chlorine, bromine or iodine; to perform Wittig Reaction, obtaining a compound represented by the following formula (5), a compound represented by the following formula (6), or a mixture of both: 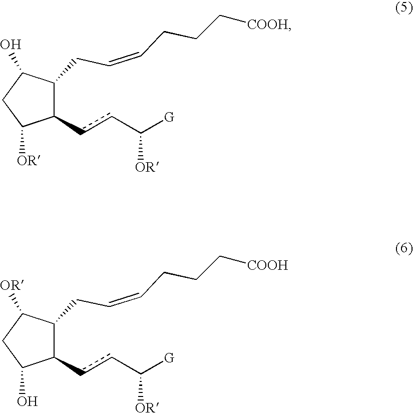
wherein G, R′ and [symbol] are as defined in claim 1; and
(d) reacting the compound represented by formula (5), (6) or the mixture of both with the following silylation agent:
Me₃Si—X
wherein X is fluorine, chlorine, bromine or iodine; to perform protection reaction, obtaining the compound represented by formula (7), (8) or the mixture of both.

3. The method of claim 1, wherein R₁ is hydrogen or isopropyl group.

4. The method of claim 1, wherein G is selected from a group consisting of:

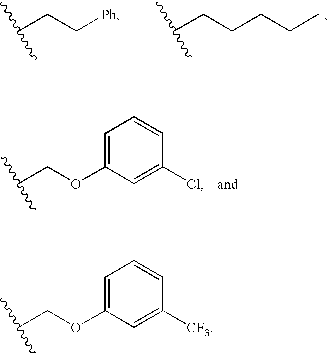

5. The method of claim 1, wherein the formula (I) is:

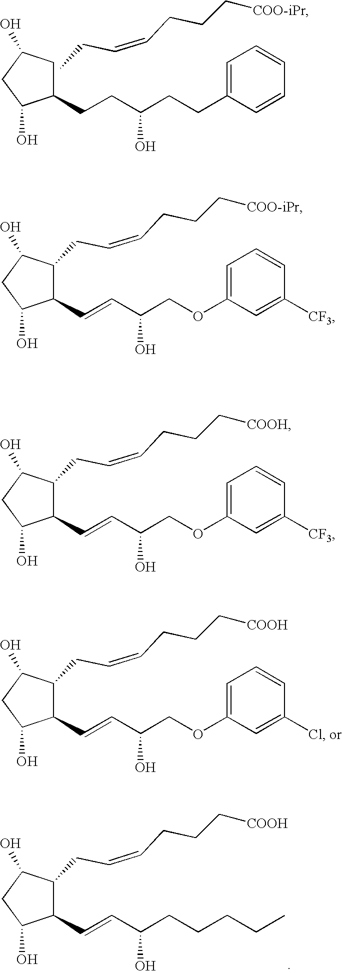

6. A compound represented by the following formula (7) or (8): 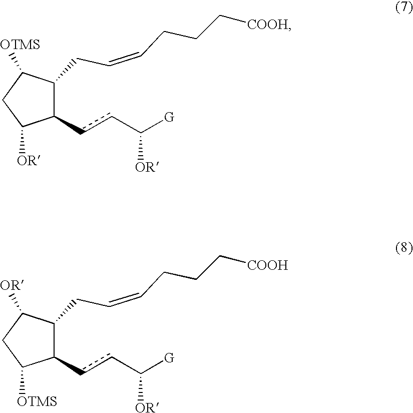
wherein,
G is selected from (i) C1-C5 linear alkyl group, (ii) —(CH)₂Ph, and (iii) —CH₂OR^{b}, wherein R^{b} is Cl- or CF₃-substituted benzyl group;
R′ in both formulas is identical and represents the following substitution group: 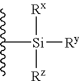
wherein R^{x}, R^{y}, and R^{z} being identical to each other or not, each independently represents C₁-C₆ alkyl group, C₆-C₁₀ aryl group, or C₇-C₁₆ arylalkyl group, while at least one of R^{x}, R^{y}, or R^{z} is not methyl group; [symbol]
represents single-bond or double-bond structure, and when being double-bond, it includes both cis- and trans-structure.

7. The compound of claim 6, wherein the compound represented by formula (7) is a compound represented by the following formula (7a): 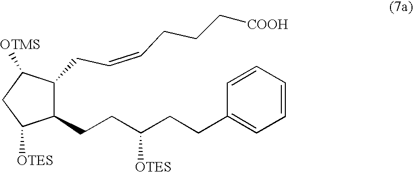
wherein TMS is trimethyl silyl; TES is triethyl silyl.

8. The compound of claim 6, wherein the compound represented by formula (8) is a compound represented by the following formula (8a): 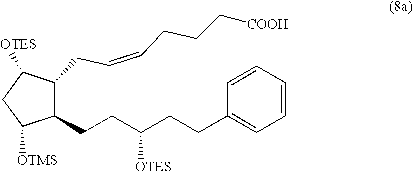
wherein TMS is trimethyl silyl; TES is triethyl silyl.

9. A compound represented by the following formula (9) or (10): 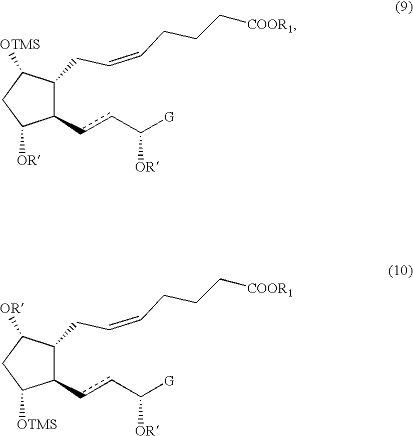
wherein,
G is selected from (i) C1-C5 linear alkyl group, (ii) —(CH)₂Ph, and (iii) —CH₂OR^{b}, wherein R^{b} is Cl- or CF₃-substituted benzyl group;
R′ in both formulas is identical and represents the following substitution group: 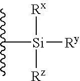
wherein R^{x}, R^{y}, and R^{z} being identical to each other or not, each independently represents C₁-C₆ alkyl group, C₆-C₁₀ aryl group, or C₇-C₁₆ arylalkyl group, while at least one of R^{x}, R^{y}, or R^{z} is not methyl group;
R1 represents hydrogen or C₁-C₅ alkyl group; [symbol]
represents single-bond or double-bond structure, and when being double-bond, it includes both cis- and trans-structure.

10. The compound of claim 9, wherein the compound represented by formula (9) is a compound represented by the following formula (9a): 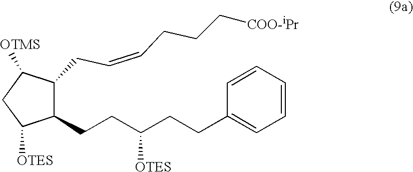
wherein TMS is trimethyl silyl; TES is triethyl silyl.

11. The compound of claim 9, wherein the compound represented by formula (10) is a compound represented by the following formula (10a): 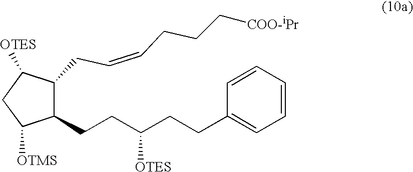
wherein TMS is trimethyl silyl; TES is triethyl silyl.
